(19) United States
(12) Patent Application Publication
(10) Pub. No.: US 2007/0122392 A1
(43) Pub. Date: May 31, 2007

(54) Propagation of undifferentiated embryonic stem cells in hyaluronic acid hydrogel

(76) Inventors: Sharon Gerecht-Nir, Brookline, MA (US); Jason A. Burdick, Philadelphia, PA (US); Gordana Vunjak-Novakovic, New York, NY (US); Robert Langer, Newton, MA (US)

Correspondence Address: CHOATE, HALL & STEWART LLP, TWO INTERNATIONAL PLACE, BOSTON, MA, 02110, US

(21) Appl. No.: 11/473,870

(22) Filed: Jun. 22, 2006

Related U.S. Application Data
(60) Provisional application No. 60/692,915, filed on Jun. 22, 2005.

(51) Int. Cl. A61K 48/00; C12N 5/08

(52) U.S. Cl. 424/93.21; 435/325; 435/366; 435/404

(57) ABSTRACT

Embryonic stem cells are propagated in a hyaluronic acid.

This application claims priority to U.S. Provisional Application No. 60/692,915, filed Jun. 22, 2005, the entire contents of which are incorporated herein by reference.

This invention was made with support from the National Institutes of Health, K22 DE-015761, P41 EB002520-1A1 and 1R01HL076485-01A2. The U.S. government may have certain rights in this invention.

### FIELD OF THE INVENTION

This invention relates to in vitro methods for promoting the propagation of embryonic stem cells.

### BACKGROUND OF THE INVENTION

Monolayer culture on a mouse or human feeder layer, Matrigel (an animal basement membrane preparation extracted from Engelbreth-Holm-Swarm mouse sarcoma), laminin, fibronectin, and in human serum are common methods available today for the propagation of undifferentiated hESCs^{1,3,4,6}. While these substrates have enabled much progress in HESC research, concerns remain about their undefined composition, variability between batches, and the hazard of zoonosis transmitted from materials of animal origin. Additionally, a cell monolayer is distinctly different from the 3D architecture of a developing blastocyst, where hESCs are embedded in an extracellular matrix (ECM), which in turn regulates their growth and differentiation^{7,8}. Thus, it is a desirable to promote HESC propagation in a 3D environment.

PCT Publication WO/2006/033103 discloses the use of hyaluronic acid-laminin gels to maintain populations of embryonic stem cells in vitro. However, cells encapsulated in these matrices divide into cells exhibiting different morphologies, e.g., endothelial-like cells and epithelial-like cells. Thus, it is desirable to provide a matrix in which proliferating cells maintain the same morphology and phenotype.

### SUMMARY OF THE INVENTION

In one aspect, the invention is a composition including a biocompatible matrix including cross-linked hyaluronic acid and mammalian embryonic stem cells disposed within the biocompatible matrix. The composition is substantially free of laminin. The composition may further include a biocompatible aqueous solvent. The mammalian embryonic stem cells may be human embryonic stem cells. The hyaluronic acid may be cross-linked through methacrylate moieties or through acrylate, thiol, or amine groups, or through biotin-streptavidin interactions. A density of cells in a composition may be from about 5 million cells/ml to about 10 million cells/ml. At least 80% of the embryonic stem cells may express one or more of tumor-rejecting antigen, stage specific embryonic antigen-4, and Oct 4. At most, 10% of the embryonic stem cells may express one or more of CD31, alpha-fetoprotein, and tubulin. The cells encapsulated within the biocompatible matrix may maintain a stable phenotype in culture for at least 30 doublings, 30 days, or 40 days. The biocompatible aqueous solvent may be culture media.

In another aspect, the invention is a biocompatible matrix consisting essentially of cross-linked hyaluronic acid, mammalian embryonic stem cells disposed within the biocompatible matrix, and a biocompatible aqueous solvent, for example, culture media.

In another aspect, the invention is a composition including a biocompatible matrix comprising cross-linked hyaluronic acid, mammalian embryonic stem cells disposed within the biocompatible matrix, and a biocompatible aqueous solvent, for example, culture media. The concentration of the hyaluronic acid in the solvent is greater than about 1.5% by weight, for example, greater than about 2%, 3%, 4%, 5%, 6%, 7%, 8%, 9%, or 10% by weight.

In another aspect, the invention is a method of culturing embryonic stem cells. The method includes providing a population of embryonic cells, combining the embryonic stem cells with hyaluronic acid to form a mixture, and causing the hyaluronic acid to cross-link in a solvent, thereby encapsulating the embryonic stem cells in a hyaluronic acid hydrogel. The encapsulated embryonic stem cells may be cultured in in vitro. The embryonic stem cells may be maintained in culture for at least 30 days, at least 40 days, or at least 30 doublings while maintaining a stable phenotype. Causing may include promoting radical chain polymerization, ionic chain polymerization or step polymerization. The method may further include allowing the cells to proliferate, releasing the cells from the hydrogel, dividing the cells into a plurality of populations, and repeating the method.

In another aspect, the invention is a method of producing a population of embryonic stem cells. The method includes providing a population of mammalian embryonic stem cells, combining the embryonic stem cells with methacrylate-terminated hyaluronic acid, causing the hyaluronic acid to cross-link in a solvent, thereby encapsulating the embryonic stem cells in a hyaluronic acid hydrogel, and contacting the hydrogel with hyaluronidase to release the embryonic stem cells.

### BRIEF DESCRIPTION OF THE DRAWING

The invention is described with reference to the several figures of the drawing, in which,

FIG. 1: HA plays a role during hESC culture on MEFs. A. Mouse embryonic fibroblasts (MEFs) secreted HA into culture medium, at concentrations that were over eight times higher than those measured for normal hESC growth medium. B. Staining of H1 hESCs grown on MEFs for HA binding site (green), undifferentiated membrane marker-TRA-1-81 (red) and nuclei (blue), revealed: (i & ii) intracellular localization of HA, including (iii) perinuclear areas (arrows) and nuclei (asterisks), as well as (iv) nucleoli (arrowheads). C. The majority of undifferentiated hESCs were found to express HA receptors CD44 (82%; middle) and CD168 (90%; right). D. i-ii. Using immunofluorescence staining, undifferentiated HESC colonies were easily detected using undifferentiated cell markers Oct4 (green) and CD44 or CD168 (red) respectively (nuclei —blue). iii-iv. Higher magnification revealed intracellular expression of CD44 and either membrane or intracellular expression of CD168.

FIG. 2: Encapsulation in HA hydrogels supported hESC viability and propagation. A. Undifferentiated H9 hESCs were passaged and re-cultured on feeder layers for 4 days in culture medium containing:. (i) no macromer, (ii) 10 μ/ml macromer, or (iii) 50 μl/ml macromer. Toxic effects were detected only at the macromer concentration of 50 μl/ml (some damaged cells marked with arrowheads). (iv) XTT assay revealed no effect of macromer on cell viability at a concentration of 10 μl/ml and a slight decrease in HESC viability at a macromer concentration of 50 μl/ml. Results are presented with ±SD (*P<0.05). B-C. Colony arrangement of undifferentiated cells detected using light microscopy at low and high magnification, respectively. D-E. Incubation with XTT revealed orange dye in viable H13 hESCs. F-H. Histology sections of H9 HESC-HA constructs cultured for 20 days demonstrate typical morphology (H&E stain) of undifferentiated colonies within 3D networks. I. i-ii Fluorescence staining of H9 HESC-HA constructs cultured for 25 days demonstrates the presence of undifferentiated hESCs. J. Staining for Ki-67 reveled that the majority of cells were proliferating. K. Caspase-3 expression was rare and L. could be observed mainly in whole colonies undergoing apoptosis. Bars-A-E,I, K−L=100 μm; F−H, J=25 μm.

FIG. 3. hESC growth rate in HA vs. Matrigel. H9 hESCs were removed from the feeder layer and cultured in the same cell concentration (per area) either within a HA hydrogel or on Matrigel, in MEF conditioned medium. XTT assay show comparable rate growth during the first 4 days of culture in both systems. Results are presented with ±SD.

FIG. 4: Cell release from hydrogels and cell karyotyping. H13 hESCs grown on MEFs were incubated for 24 hr in A. growth medium; B. 1% collagenase in growth medium; C. 1000 U/ml, and D. 2000 U/ml hyaluronidase in growth medium. To release hESCs from HA hydrogel, constructs were incubated 2000 U/ml hyaluronidase in growth medium: E. After 18 hr, small particles of hydrogels remained that trapped hESCs. F. After 24 hr, hESCs colonies were completely released from the hydrogel. G. H9 hESCs released from the hydrogel were cultured on MEFs and formed small undifferentiated colonies after 24 hours. H. H9 hESCs released from hydrogels were propagated on MEFs for 3 passages. Genetic integrity was further examined and abnormalities events could not be detected in: I. H9 p22 grown on MEFs, J. H9 p22 grown on MEFs and exposed to UV for 10 min, K. H9 p22 grown on MEFs and incubated with growth medium containing 2000 U/ml hyaluronidase. L. H9 p38 removed from MEFs and encapsulated in HA hydrogels for 5 days followed by incubation with growth medium containing 2000 U/ml hyaluronidase for 24 hand re-culture on MEFs for 3 passages. Bars=100 μm

FIG. 5. EB differentiation. H13 hESCs encapsulated in HA hydrogel for 30 days were released and cultured in suspension to allow EB formation. Histology sections of 30 days old EBs revealed A. typical EB organization with B. detectable remains of HA entrapped within the bodies (arrows). C. Various cell types and cellular organization could be observed using higher magnification.

FIG. 6. Genetic integrity. Karyotyping of hESC H13 line was also evaluated in: A. H13 p25 grown on MEFs, B. H13 p25 grown on MEFs and incubated with growth medium containing 2000 U/ml hyaluronidase, and C. H13 p25 grown on MEFs and exposed to UV for 10 min. Abnormality events could not be detected in any of the conditions.

FIG. 7: HA Internalization and degradation. A. Encapsulation of H13 hESCs in HA hydrogels was compared to dextran hydrogels after 15 days of culture. Light microscope images of both cultures at low and high magnifications and H&E staining of sectioned gels demonstrate embryoid body formation in dextran gels vs. colony arrangements of undifferentiated hESCs in HA gels. B. Undifferentiated H1 hESCs grown on MEFs were incubated overnight with fluorescein-HA, and further stained for CD44 and CD168. HA uptake by H9 hESC colonies: i edge of colony internalizing FL-HA via CD44; ii & iii intracellular localization of FL-HA in hESC colonies C. H13 hESC colonies grown on MEFs positive for Oct4 (green) express (i) Hyal 1 or (ii) Hyal 2 (red; nuclei in blue) mainly in densely packed areas of the colonies D. RT-PCR analysis revealed high expression levels of a hyaluronidase isomer, Hyal 2, in undifferentiated H9 hESCs. PC3 line served as positive control. Bars=100 μm

FIG. 8. HA receptors in response to addition of human FL-HA. FL-HA was added to the growth medium of H9 hESCs cultured on MEFs. Confocal analysis revealed localization in cell membranes of both A. CD44 and B. CD168 (red, nuclei in blue).

### DETAILED DESCRIPTION OF CERTAIN PREFERRED EMBODIMENTS

In an exemplary embodiment, mammalian embryonic stem cells (ESC) are disposed within a cross-linked hyaluronic acid matrix and cultured in appropriate media. The cells remain undifferentiated in vitro for extended periods of time. The ESC may be human or non-human ESC.

In one embodiment, ESC were encapsulated in a hydrogel scaffold that is composed of biologically recognized molecules. Hydrogels were selected because they not only have a high water content to promote cell viability, but they are structurally and mechanically similar to the native ECM of many tissues⁹. HA, a nonsulfated linear polysaccharide of (1-β-4) D-glucuronic acid and (1-β-3) N-acetyl-D-glucosamine, was selected because it co-regulates gene expression, signaling, proliferation, motility, adhesion, metastasis, and morphogenesis¹⁰. Notably, HA content is greatest in undifferentiated cells and during early embryogenesis and then decreases at the onset of differentiation¹¹. In spite of its known role in embryogenesis^{10,11}, HA has not been extensively utilized for the cultivation of hESCs.

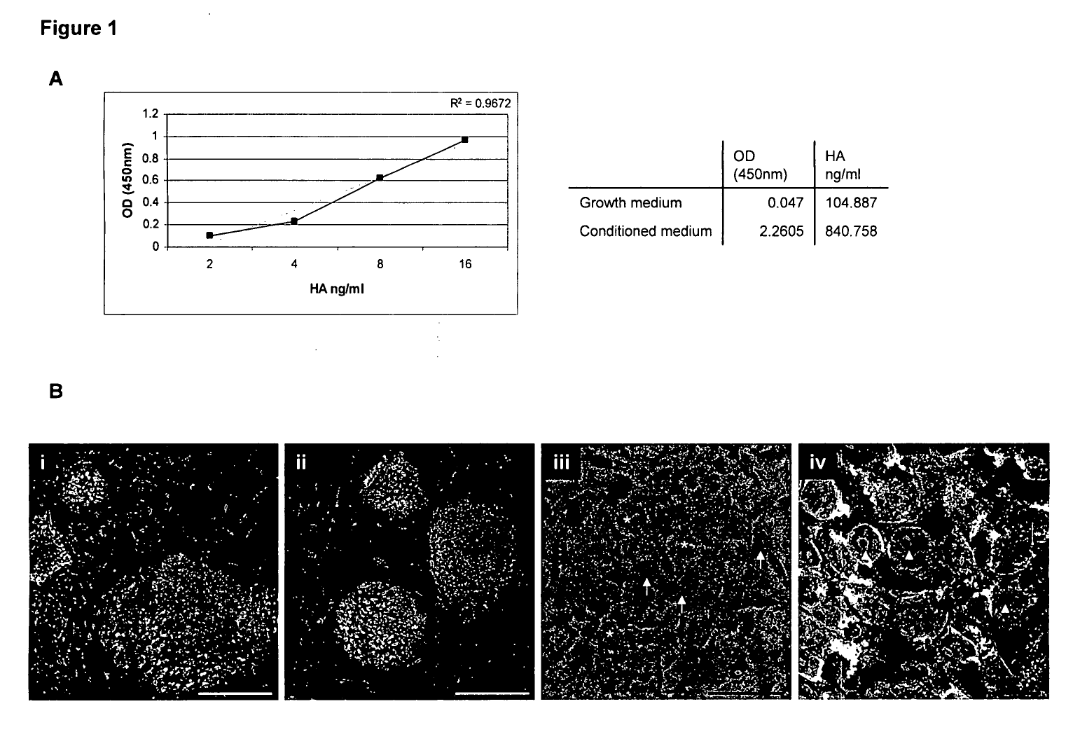

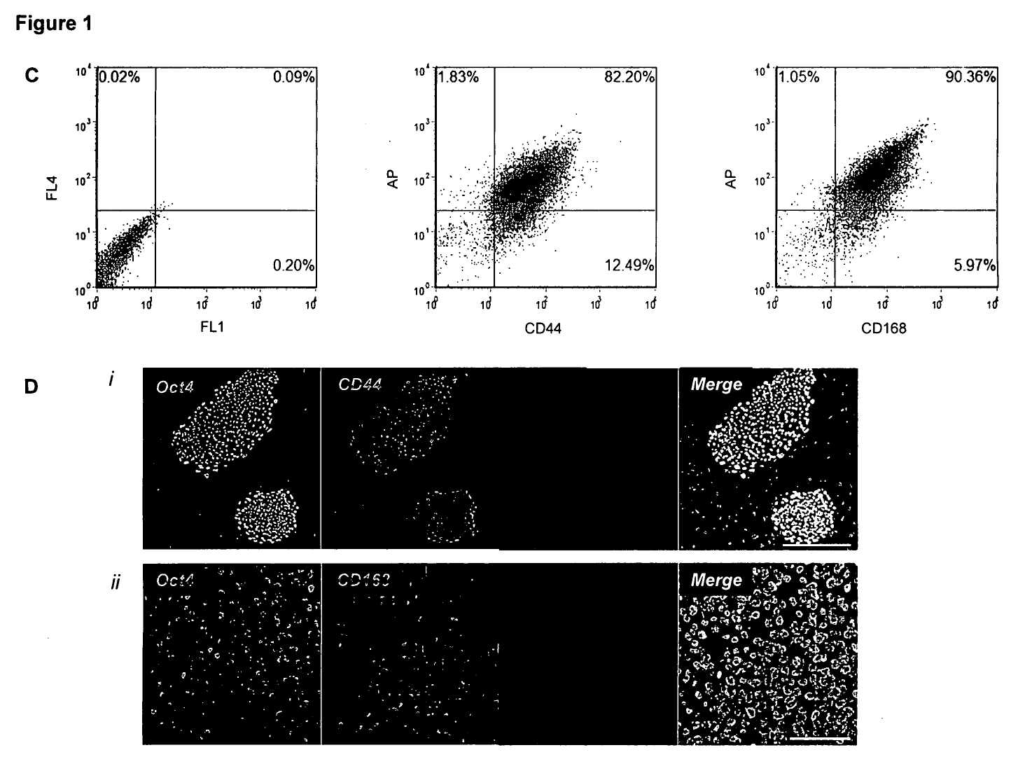

We observed that mouse embryonic fibroblasts (MEFs) that form feeder layers for HESC cultivation produce high levels of HA (FIG. 1A), and that abundant HA binding sites were located intracellularly in undifferentiated hESCs (FIG. 1B). This is consistent with previous evidence that HA is localized intracellularly, in endosomes and perinuclear tubular vesicles¹², rough endoplasmic reticulum¹⁴, nuclei and nucleoli¹⁴. Also, without being bound by any particular theory, the success of mouse feeder layers for the cultivation of hESCs might be related to their ability to secrete HA.

During development, cellular interactions with HA are mediated at least in part by CD44 and CD168. CD44 is a mediator for HA-induced cell proliferation and survival pathways¹⁰ and is present in human cumulus cells, oocytes, early embryos and pre-hatched blastocysts¹⁵. CD44 is also involved in the initial binding of HA to the cell surface prior to its internalization and degradation by acid hydrolases. CD168 is involved in HA-induced cell locomotion¹⁶, and its expression in early embryos was recently documented¹⁷. During in vitro culture, undifferentiated hESCs expressed high levels of CD44 and CD168 (FIG. 1C). In fact, hESC colonies cultured on MEFs could be easily visualized by staining for CD44 (FIG. 1Di) or CD168 (FIG. 1Dii). Undifferentiated cells were characterized by intracellular expression of CD44 (FIG. 1Diii) and either membrane or intracellular expression of CD168 (FIG. 1Div).

Various attempts to culture HESC monolayers on a HA substrate resulted in low efficiency of cell adhesion (<15%), consistent with low adhesion of other cells expressing HA receptors such as NIH3T3¹⁹. We therefore elected to encapsulate ESCs within HA, in a manner that combines cell scaffolding with the use of a chemically defined system. We further selected HA hydrogels because they allow gentle entrapment of differentiated mammalian cells without a loss of their viability²⁰. In our previous studies, a HA hydrogel fabricated from a 2 wt % solution of a 50 kDa macromer supported the highest viability of differentiated mammalian cells²⁰. One advantage of HA hydrogels is that the chemistry of the network is easily controlled via reaction conditions and is uniform between different batches²⁰, in contrast with naturally derived matrices such as Matrigel.

To form hydrogels, HA may be chemically modified with methacrylate groups that, in the presence of light and a photoinitiator, undergo a free-radical polymerization. Exemplary initiators include but are not limited to 2-methyl-1-[4-(hydroxyethoxy)phenyl]-2-methyl-1-propanone (Irgacure 2959, 12959), a photoinitiator, and thermal and redox initiation systems such as those employed for methacrylate bone cements. The initiator may be optimized to minimize its chemical influence on the encapsulated cells and to minimize any effect that the initiation conditions may have on the cells. Other functional groups that may be used to crosslink the HA are familiar to those of skill in the art and include but are not limited to acrylates, amines, and thiols. While light-initiated radical polymerization may provide simpler initiation and reaction conditions, other polymerization mechanisms, e.g., thermal or other radical initiation conditions, ionic chain polymerization or step polymerization, may be employed as well. One skilled in the art will be familiar with appropriate reactive groups, initiators, etc. for forming cross-linking polymers using these methods. Further examples may be had by reference to Odian, Principles of Polymerization, Fourth Edition, Wiley Interscience, 1994, the entire contents of which are incorporated by reference. HA may also be functionalized with biotin or streptavidin and crosslinked through streptavidin-biotin interactions.

For encapsulation, ESCs are suspended in a solution of HA macromer and polymerized into a network. Exemplary molecular weights of the HA macromer range from about 5 kDa to about 2000 kDa, for example, about 50 kDa, about 350 kDa, or about 1100 kDa. Exemplary molecular weights may range from about 5 kDa to about 50 kDa, from about 50 kDa to about 100 kDa, about 100 kDa to about 500 kDa, about 500 kDa to about 1000 kDa, about 1000 kDa to about 1500 kDa, or about 1500 kDa to about 2000 kDa. The crosslink density of the resulting gels may be correlated to the initial concentration of the HA (wt %) in the precursor solution. Exemplary concentrations may range as low as 0.5%, for example, about 0.5% to about 1%, about 1% to about 2%, about 2% to about 4%, about 4% to about 6%, about 6% to about 8%, about 8% to about 10%, or even greater. While concentrations as high as 40% may be achievable, one of skill in the art will recognize that the concentration may be optimized to maximize cell viability while maintaining the structural integrity of the hydrogel as the cells propagate.

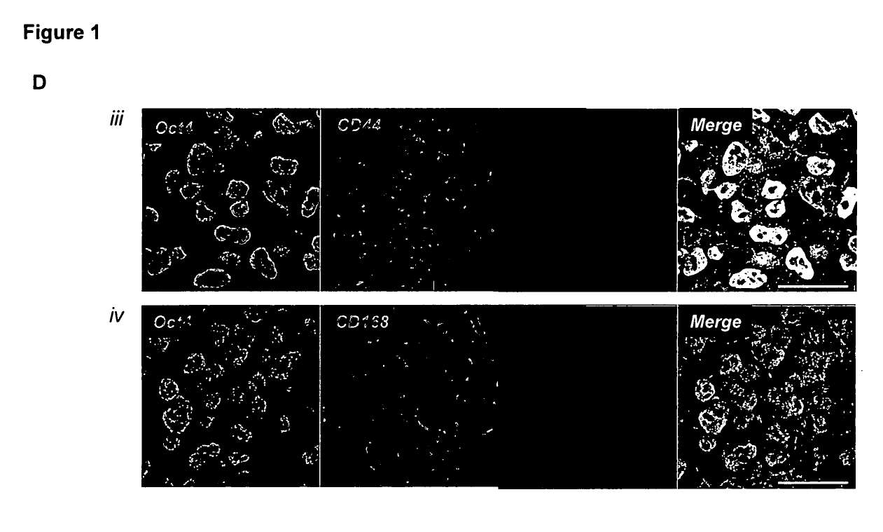

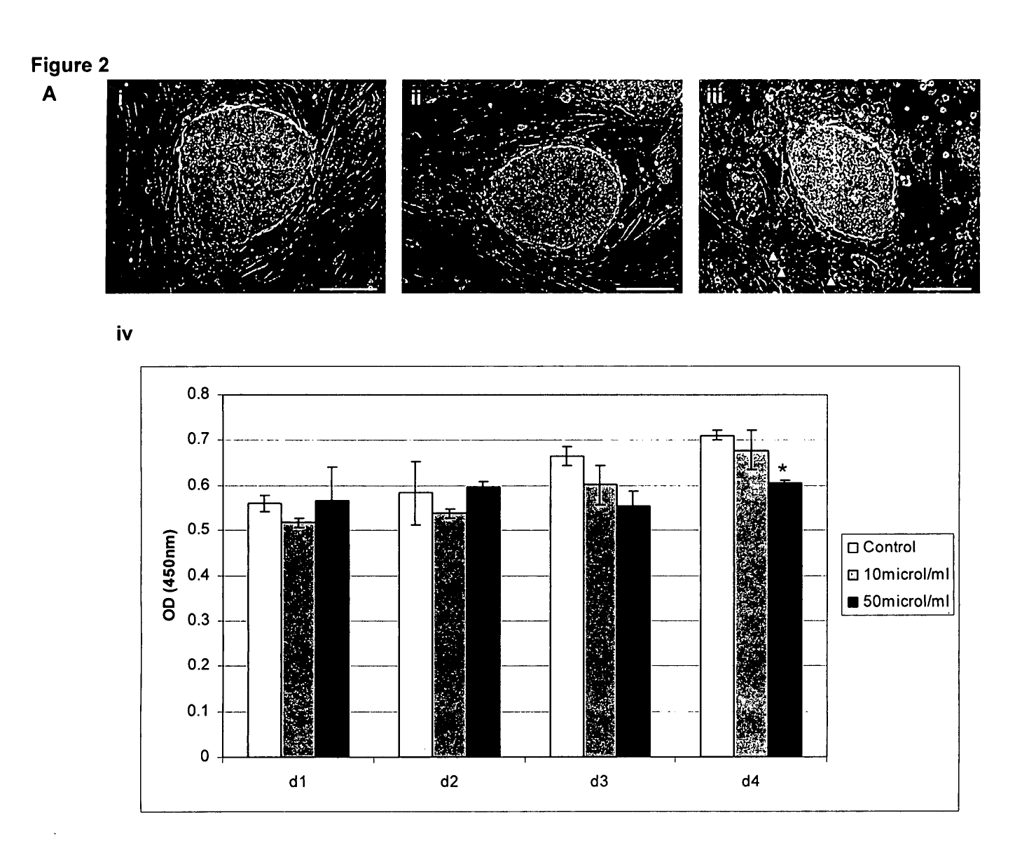

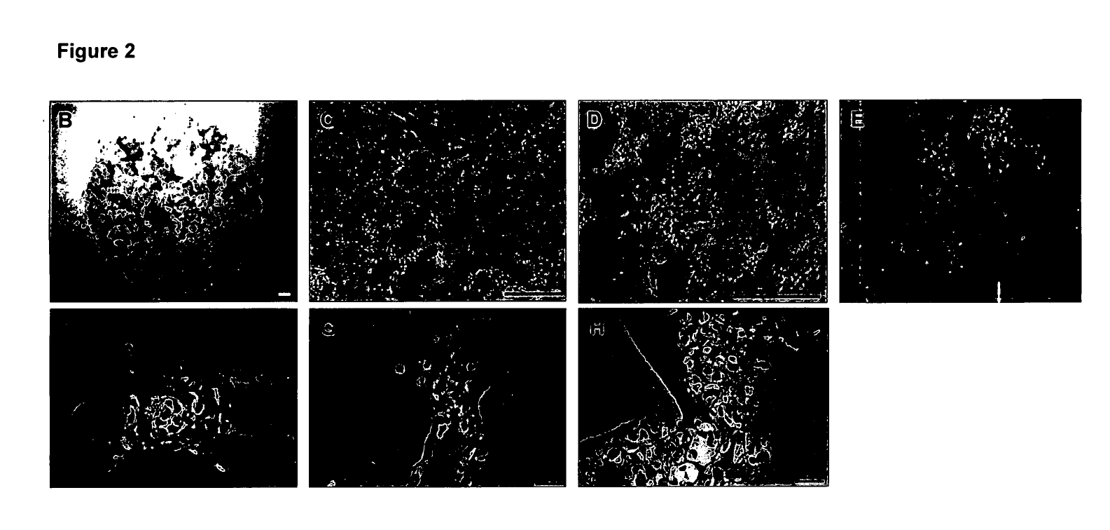

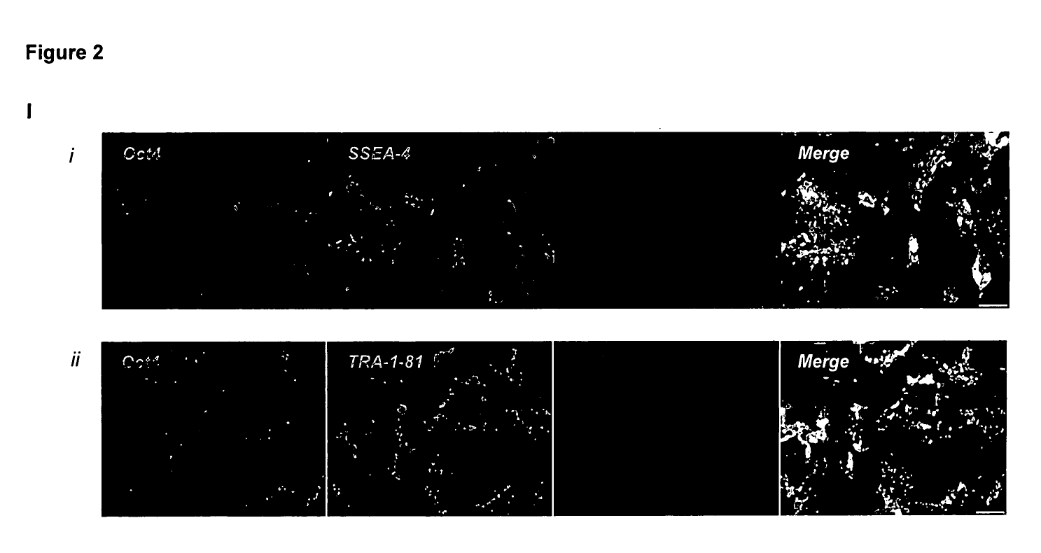

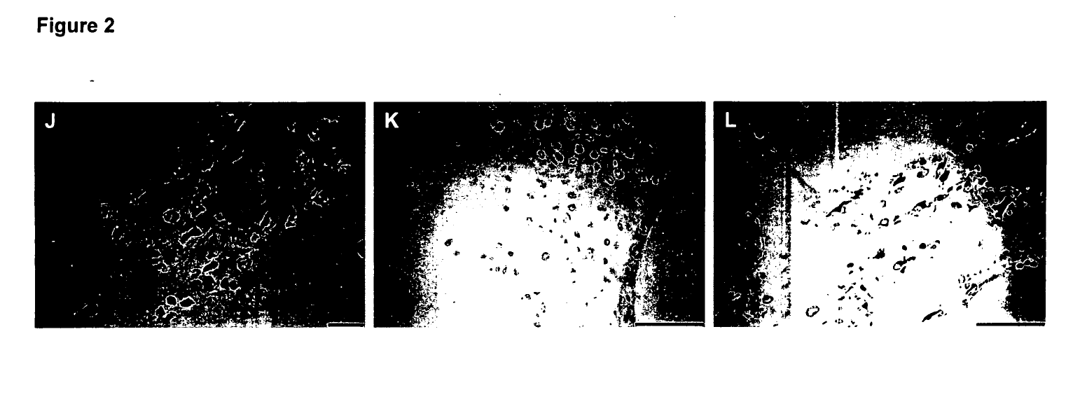

Exemplary hydrogels fabricated with 50 kDa HA contained spatially uniform cell distributions (FIG. 2B-C). The viability of hESCs was maintained throughout the 25 days of cultivation, as demonstrated by XTT staining (FIG. 2D-E). A typical undifferentiated morphology was observed in hESC colonies within the HA networks (FIG. 2F-H). High cell concentrations, in the range of 5 to 10 million cells per ml of the precursor solution, were optimal for high viability and sustained growth of hESCs. At hESC concentrations greater than ten million cells/ml, large clumps of cells formed that underwent rapid apoptosis, whereas cell concentrations lower than 5 million cells per ml could not support colony formation within the networks (data not shown). The same phenomenon of colony concentration-dependence of hESC culture is well recognized for 2D monolayers^{22,23}. Early in culture, the growth rates of hESCs within HA hydrogel and on Matrigel were comparable (FIG. 3).

Exemplary HESC populations (see examples) were propagated in gels formed from 50 kDa HA for up to 30 doublings (˜40 days); further expansion may depend on the hydrogel structure. For more loosely crosslinked hydrogels (e.g., 1 wt % solutions of macromer), the gel does not maintain its structural integrity past this point. With more densely crosslinked hydrogels (e.g., 2 wt % solutions), the cell expansion rates are hindered. One skilled in the art will recognize that the concentration of macromer in solution may be adjusted to optimize the propagation rate and the number of doublings. After a certain period of time, cells may have proliferated sufficiently that there is no more room for further cell proliferation. This time period will vary with crosslink density but is around 20 days for 2 wt % solutions of 50 kDa HA. At this point, proliferation may be continued by releasing the cells from the HA gel and re-encapsulating them. The optimal frequency of release and re-encapsulation (e.g., “3D passaging”) depends in part on the original seeding density and the molecular weight and cross-link density of the HA. In some embodiments, cells may be passaged every 10 days, every 15 days, every 20 days, or at some other frequency.

In one embodiment, the developing HESC colonies expressed high levels of stem cell markers after more than 30 days of culture, including the tumor rejecting antigen (TRA)-1-81 (˜93%), stage specific embryonic antigen-4 (SSEA-4) (˜98%), and Oct 4 (˜97%); (FIG. 21). For example, at least 80%, at least 85%, at least 90%, or at least 95% of ESC in culture may express one of these markers, indicating that the cells are maintaining the stem cell phenotype. For cells cultured according to an exemplary embodiment of the invention, differentiation markers for mesoderm (CD31), endoderm (α-fetoprotein) and ectoderm (tubulin) were not detected. In some embodiments, at most 10%, at most 5%, or at most 1% of the ESC express one or more of these markers.

The maintenance of cellular viability within the hydrogel constructs was documented by the presence of an array of markers. The human Ki-67 protein, which is present during all active phases of the cell cycle, was expressed by the majority of encapsulated hESCs (58±5%) (FIG. 2J). Only occasional apoptotic events could be observed using a Tunnel assay (14±3%). These results correlate to a recent study which showed that >50% of the cell nuclei within hESC colonies grown on MEFs are in a proliferating phase²⁴. Only rare expression of caspase-3, a marker activated in cells undergoing apoptosis, was found within HA-HESC constructs (3±8%). When detected, caspase-3 appeared in a whole colony rather than in single cells within different colonies (FIG. 2K-L). Therefore, under the conditions studied, diffusion of nutrients and oxygen to the cells through the 2 wt % HA hydrogel appeared to be rapid enough to support normal cell growth rates.

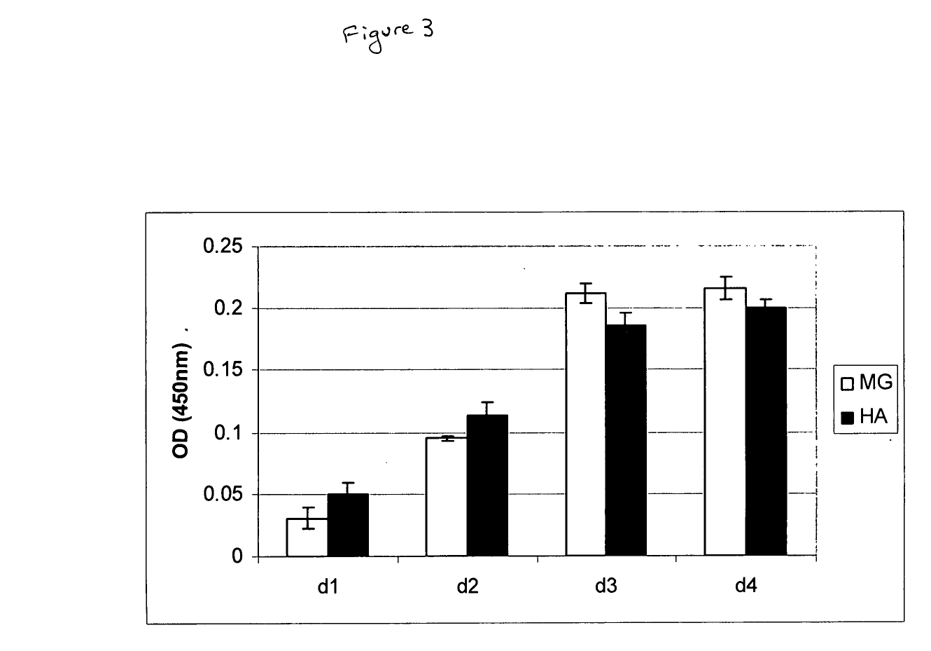

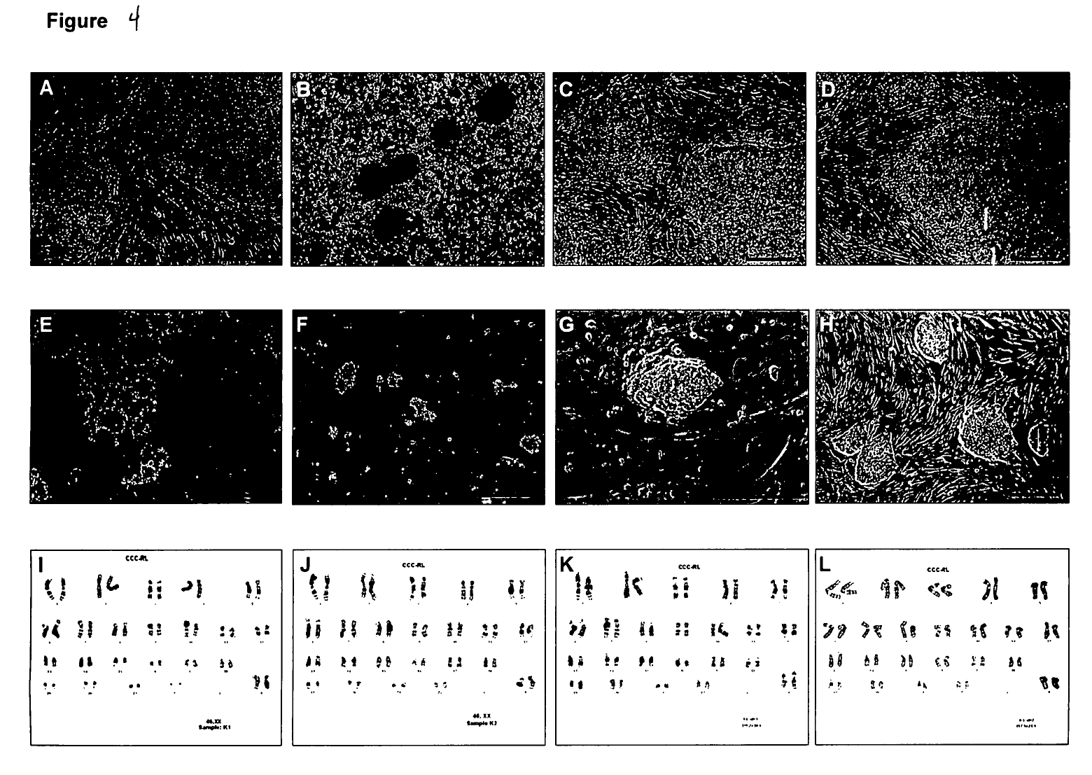

To use the ESC for research and cellular therapy, the cells may need to be released from the hydrogel. An exemplary method for releasing the cells is by treating the HA hydrogel with hyaluronidase²⁰ at a concentration of about 500 to about 2000 U/mL. We examined hESCs to determine if they survive long-term treatment (i.e., 24 hours) with hyaluronidase. HESC colonies incubated with growth medium containing hyaluronidase at all concentrations preserved their normal morphology with no apparent loss of viability (FIG. 4A-D). We found that hyaluronidase concentrations of <1000 U/ml resulted in only partial degradation of HA hydrogels over a 24 hr period, and were associated with low efficiencies of HESC retrieval. Incubation of HA-HESC constructs in growth medium containing 2000 U/ml hyaluronidase resulted in complete degradation of the hydrogel (FIG. 4E-F). One of skill in the art will recognize that the optimal concentration and incubation time may vary depending on the crosslink density and molecular weight. Lower concentrations may also be employed where it is desirable to study the gel after the cells are released. Colonies released from the gels readily adhered to the MEF (FIG. 4G) with high adherence efficiency (80% after 48 hours) and proliferated for at least 5 passages without the differentiation that is often seen in standard monolayer cultures of hESCs (FIG. 4H).

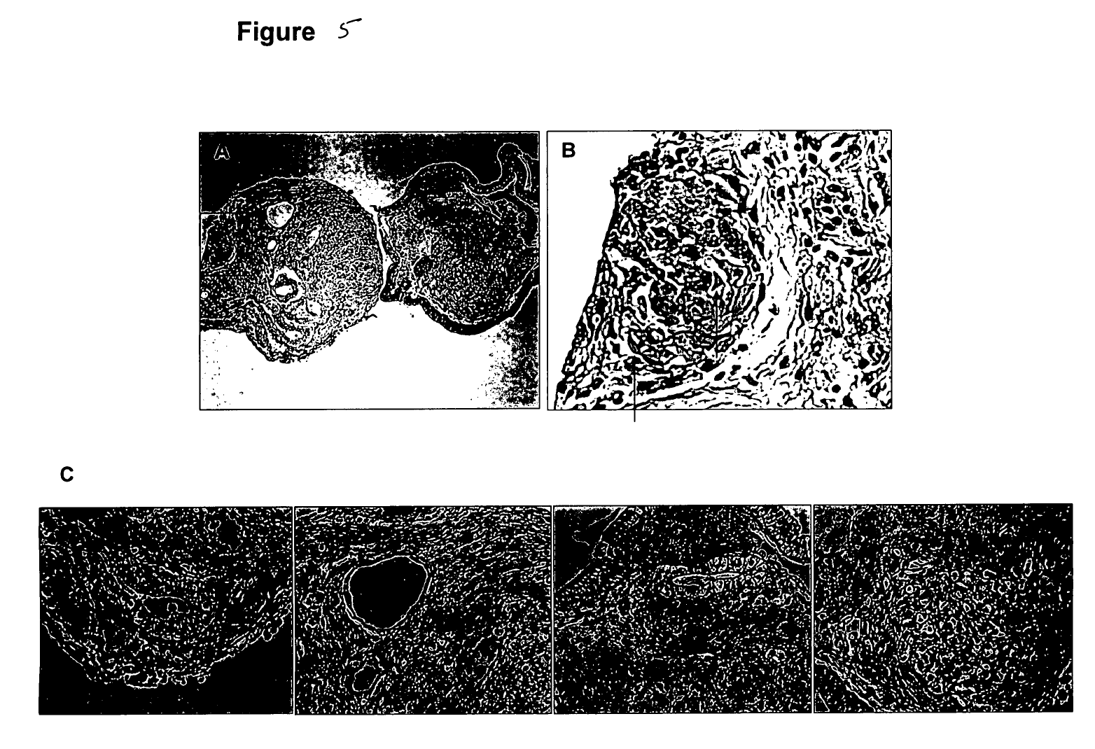

Importantly, the release of hESCs from the HA hydrogels was associated with the preservation of cell viability and undifferentiated state. In correlation with the in situ staining, FACS analysis showed high levels of expression of stem cell marker SSEA-4 (96%) and minimal levels of the differentiation marker CD31 (<0.3%) (data not shown). To demonstrate that the HA-borne hESC colonies maintained their pluripotency, the differentiation potential of the cells released from HA hydrogels was examined by the spontaneous formation of embryoid bodies (EBs). hESCs encapsulated in HA hydrogels for 35 days, released using hyaluronidase and cultured in suspension formed EBs containing various cell types (FIG. 5).

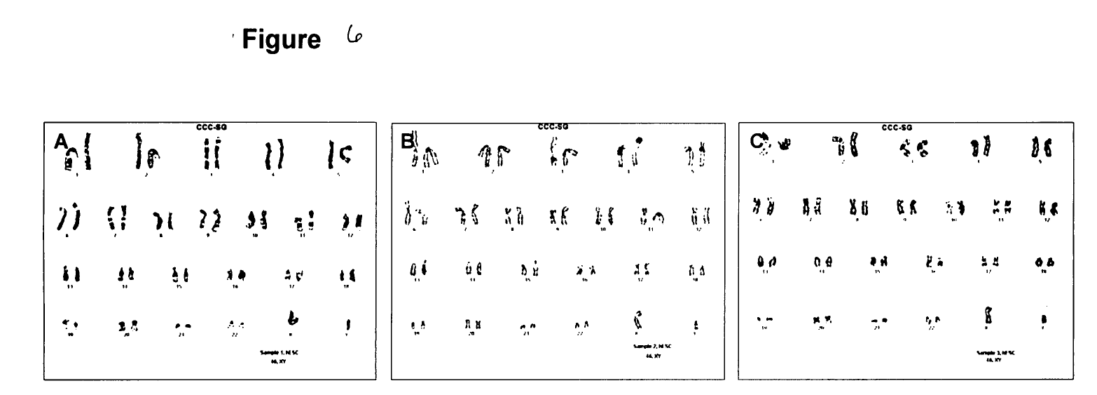

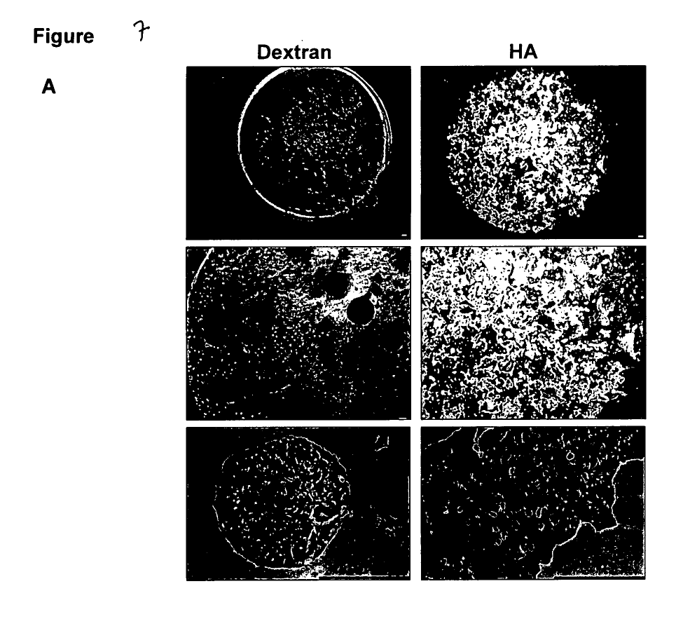

The proposed system for ESC culture in a three-dimensional HA hydrogel and the release of expanded ESCs involves the exposure of ESCs to low intensity UV light (e.g., ˜10 mW/cm² for 10 min) and treatment with hyaluronidase (e.g., 2000 U/ml for 24 hours). Since these factors could potentially affect the genetic integrity of ESCs, karyotype analysis was performed on: (i) undifferentiated hESCs cultured on MEFs (H9 line p22; H13 line p25); (ii) undifferentiated hESCs cultured on MEFs (H9 line p22; H13 line p25) and exposed to UV light for 10 min; (iii) undifferentiated hESCs cultured on MEFs (H9 line p22; H13 line p25) treated with hyaluronidase (2000 U/ml) for 24 h; and (iv) undifferentiated hESCs (H9 line p38) encapsulated in HA gels for 5 days followed by their release and re-culture on MEFs for an additional 3 passages. All hESCs were found to have normal 46XX karyotyping (FIG. 4I-L and FIG. 6). Hence, the application of UV light and hyaluronidase at the levels necessary for releasing cells from HA gels appear to be safe for hESCs.

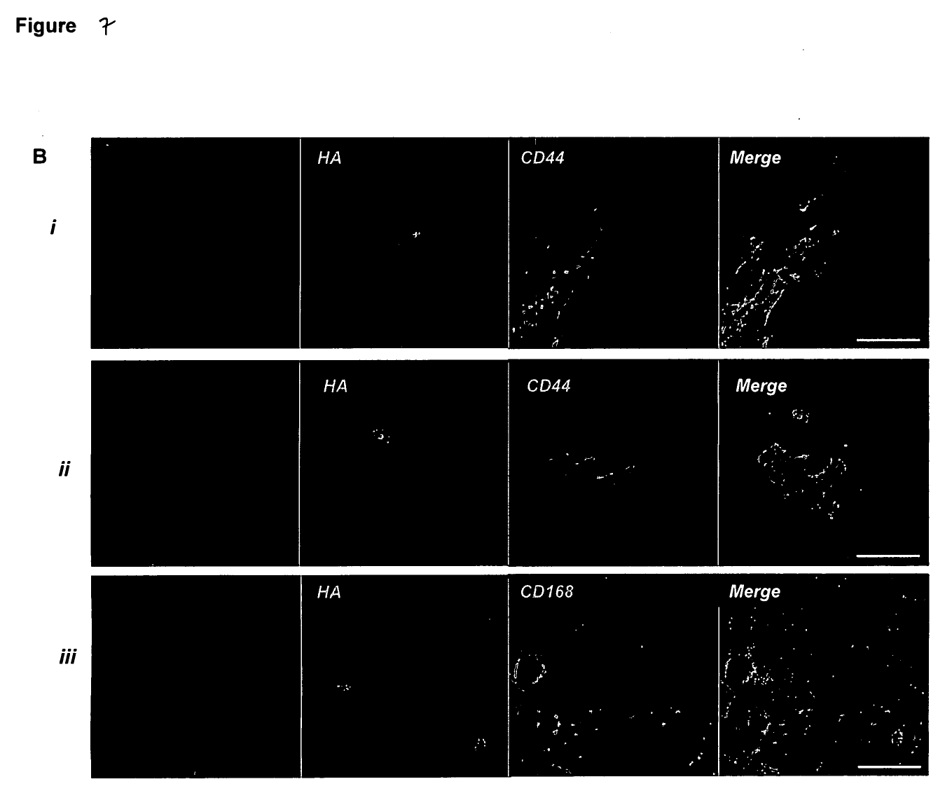

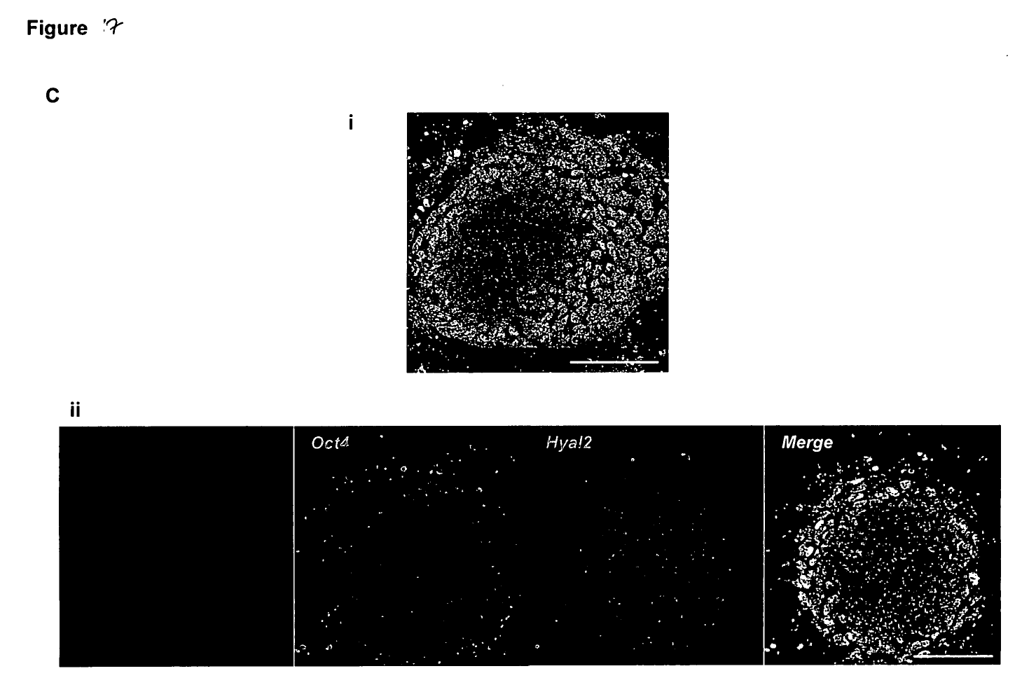

To determine the importance of ESC-HA interactions for the propagation of ESCs in their undifferentiated state, hESCs were encapsulated in networks formed from a different polysaccharide, dextran, using the exact same methodology of photopolymerization for cell encapsulation. In contrast to the proliferation of undifferentiated hESC colonies in the HA system, dextran hydrogels induced hESC differentiation and the formation of embryoid bodies (FIG. 7A). These results are consistent with data published for other hydrogel systems that also supported hESC differentiation^{25,26}. Without being bound by any particular theory, the regulatory role of HA in the maintenance of hESCs in their undifferentiated state, in vitro as well as in vivo, may contribute to the ability of hESCs to propagate in HA without differentiating.

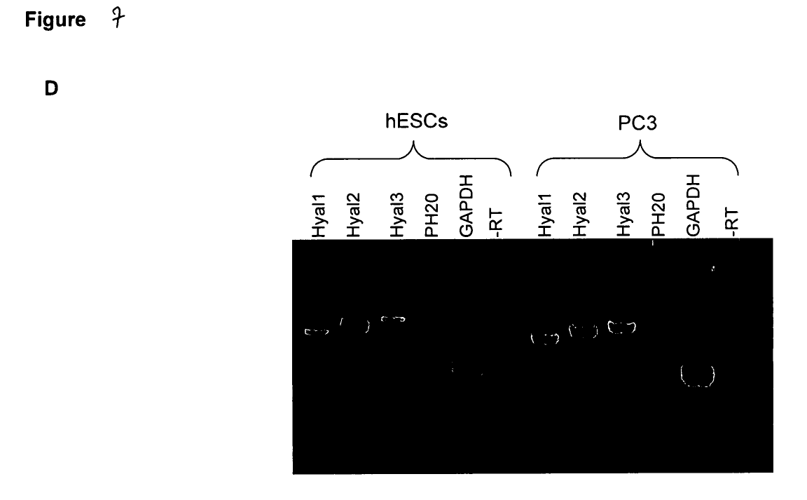

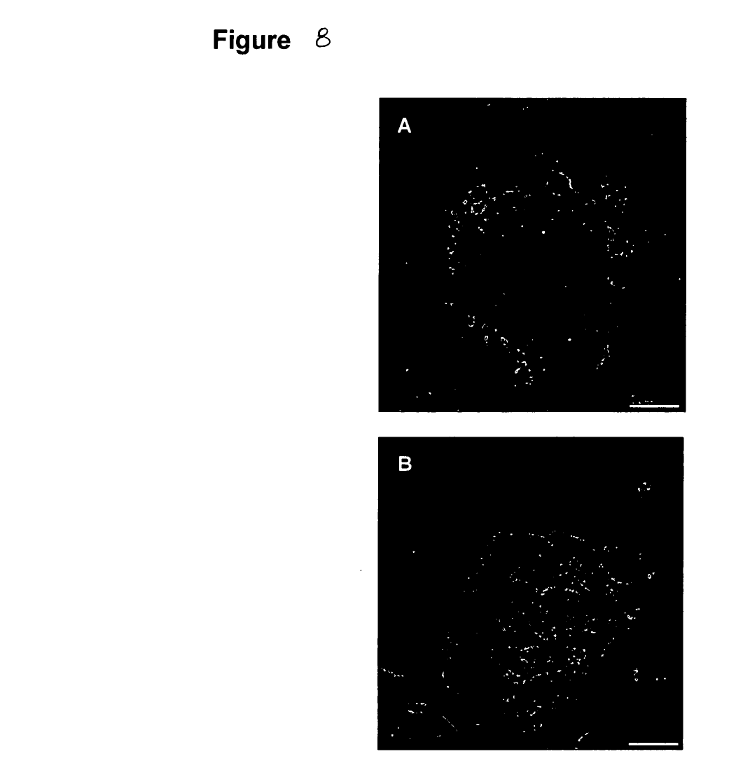

The addition of human FL-HA to the culture of hESCs on MEFs resulted in the localization of HA receptors to the cell membranes, first at the edges of cell colonies and then at their centers (see FIG. 8). FL-HA was internalized through the membrane receptors (FIG. 7Bi) and localized within the cells (FIG. 7Bii-iii), indicating receptor-mediated internalization of HA by hESCs. Immunofluorescence of HESC colonies cultured on MEF revealed that densely packed colonies expressed human hyaluronidase Hyal 1&2 (FIG. 7C). RT-PCR analysis corroborated that hESCs express high levels of expression of Hyal 2, one of the isoforms of human hyaluronidase (FIG. 7D). It was previously suggested that HA originates from the pericellular material which is degraded intracellularly^{27,28}. Without being bound by any particular theory, our data suggest that hESCs are able to uptake and degrade HA and thereby remodel HA gels, which can further promote their survival and proliferation.

These data demonstrate that viable, proliferating ESCs can be maintained in their undifferentiated state when cultured in HA hydrogels, and released without the loss of cell viability. ESC survival and proliferation were associated with the presence of developmentally relevant signals and the ability of cells to remodel the HA gel. Hydrogels formed as networks of HA fibers appear to have the ability to maintain proliferating hESCs in their undifferentiated state (in contrast to dextran networks or cell monolayers on HA) and under chemically defined conditions (in contrast to soluble HA, MEFs, Matrigel and human serum).

### EXAMPLES

hESCs. Three different lines of hESCs were studied (WA9, WA13 and WA1, obtained from WiCell Research Institute, Madison, Wis.; p19-40).

hESC culture on MEFs. hESCs were grown on inactivated mouse embryonic fibroblasts (MEFs) in growth medium including 80% KnockOut DMEM, supplemented with 20% KnockOut Serum Replacement, 4 ng/ml basic Fibroblast Growth Factor, 1 mM L-glutamine, 0.1 mM β-mercaptoethanol, 1% non-essential amino acid stock (Invitrogen Corporation, Carlsbad, Calif.). hESCs were passaged every four to six days using 1 mg/ml type IV collagenase (Invitrogen Corporation, Carlsbad, Calif.).

hESC encapsulation and release. Methacrylated HA was synthesized as previously described²⁰. Briefly, HA (50 kDa, Lifecore) was dissolved in deionized water and adjusted to a pH of 8.0 with 5 N NaOH. Methacrylic anhydride (Aldrich) was slowly added and the reaction mixture was incubated overnight at room temperature. The product was dialyzed for purification, lyophilized, and stored as a powder at 0° C. The methacrylated HA was dissolved at a concentration of 2 wt % in PBS containing 0.05 wt % 2-methyl-1-[4-(hydroxyethoxy)phenyl]-2-methyl-1-propanone (Irgacure 2959, I2959) and hESCs were added (0.5−1×10⁷ cells/ml precursor solution). The mixture was pipetted into a sterile mold (50 μL volume per well, to obtain discs measuring 3 mm in diameter×2 mm thick), and photopolymerized (˜10 mW/cm² UV light, BlakRay, for 10 min).

Dextran-acrylate macromer was prepared as described previously²⁹. Dextran (10 g) and vinyl acrylate (1.21 g) were dissolved in DMSO (150 mL) and the reaction initiated by adding 1.5 g of Proleather (enzyme from Bacillus sp.). The reaction mixture was shaken at 50° C. (250 rpm) for 72 h, and then precipitated in acetone. The precipitate was dissolved in water and dialyzed for 5 days against milli-Q water, at 420 C., and finally lyophilized. hESCs were encapsulated within the dextran using the same procedures as for HA hydrogels.

Cell-gel constructs were cultivated in hESC grwoth medium containing 100 ng/ml bFGF or MEF conditioned media as previously described¹ Briefly, hESCs growth medium was incubated on inactivated MEF for 24 hours and collected and filtrated. bFGF (4 ng/ml) was added before use. Constructs were incubated up to 40 days. To release encapsulated hESCs, HA constructs were incubated for 24 h in hESC growth medium containing 100, 500, 1000 or 2000 U/ml hyaluronidase (Sigma, St. Louis, Mo.). For re-culture, cells were collected, centrifuged, washed three times with PBS to remove any hydrogel residues, re-suspended in growth medium, and cultured on MEF coated dishes using standard methods^{22,23}. For EB formation, hESCs were cultivated in non-adherent Petri-dishes.

Presence of HA in medium. MEF conditioned medium was prepared as previously described¹ and compared to hESCs growth medium with respect to the levels of HA using a HA test kit (Corgenic, Inc., Westminster, Colo.) according to the manufacturer's instructions.

HA receptors and stem cell/differentiation markers. hESCs were removed from MEFs or released from hydrogels and filtered through a 40 μm mesh strainer (BD, San Jose, Calif.). Expression of alkaline phosphatase (AP) was considered as an indicator of undifferentiated state of hESCs. Intrastain kit (Dako California Inc. Carpinteria, Calif.) was used for the fixation and permeabilization of cell suspensions, according to the manufacturer's instructions. Briefly, dissociated hESCs were blocked with 5% FBS/PB-S, incubated with anti-human CD44 clone A3D8 (Sigma, St Louis, Mo.), or IgG antibody (R&D systems, Minneapolis, Minn.) for 30 min, and washed with PBS, followed by incubation with donkey anti-mouse FITC (Vector Labs Burlingame, Calif.) for 15 min. Cells were stained with APC conjugated anti-human AP or PE conjugated anti-human SSEZ4 (both from R&D systems, Minneapolis, Minn.) for stem cell markers or with FITC conjugated anti-human CD31 (BD, San Jose, Calif.) as marker of differentiation. The cells were washed twice prior to flow cytometry. hESCs were analyzed using FACSCalibur (BDIS) and Cell Quest software (BDIS).

Toxicity Assay

Because hESCs can be sensitive to culture conditions²¹, we assessed any toxicity of the methacrylated HA macromer. hESCs were propagated in monolayers with two concentrations (i.e., 10 and 50 μ/ml) of the HA macromer in the culture media. Colonies of hESCs were formed at all culture conditions and have continuously grown with time (FIG. 2A i-iii). Comparison of the proliferation rates revealed toxic effects only at a macromer concentration of 50 μ/ml (FIG. 2A iv), a level corresponding to completely non-polymerized HA and, therefore much higher than that seen by the encapsulated cells. The rate of cell proliferation at a macromer concentration of 10 μ/ml, a level corresponding to a HA hydrogel that was polymerized to 80% incorporation of the macromer, was indistinguishable from cells cultured in control medium (FIG. 2A iv). Radical polymerization to produce loosely crosslinked HA hydrogels occurs at high conversion rates and the release of unreacted macromer is only minimal, thus eliminating any toxicity that may result from the presence of free HA macromer.

Proliferation assay. Proliferating cells were detected by the XTT kit (Sigma, St. Louis Mo.) according to the manufacturer's instructions. Undifferentiated hESCs cultured in the presence of macromer on Matrigel and within HA cultures were incubated for 4 h in medium containing 20% (v/v) XTT solution. For analysis, 150 μl of the medium were removed, placed in a 96-plate well and read in a microplate reader at 450 nm. XTT was also used for visual analysis of viable cells within hydrogels in which HA constructs were incubated for 4 h in medium containing 20% (v/v) XTT solution and examined using Inverted light microscopy (Nikon Diaphot system).

Immunohistochemistry. HA constructs were either embedded in histo-gel or directly fixed in 10% neutral-buffered formalin (Sigma, St. Louis, Mo.) overnight, dehydrated in graded alcohols (70-100%), embedded in paraffin, sectioned to 4 μm, and stained with hematoxylin/eosin. Immunostaining was performed using a Dako LSAB®+ staining kit (Dako California Inc. Carpinteria, Calif.) with specific anti tumor rejection antibody (TRA)-1-60, anti TRA-1-81, and anti CD44 clone P3H9 (Chemicon Temecula, Calif.). Mouse IgG isotype-matching (R&D systems, Minneapolis, Minn.) or secondary antibody alone (from Dako LSAB®+ staining kit) served as negative controls. For proliferation assessment, anti-Ki67 (BD Pharmingen, San Jose, Calif.) was used. For apoptotic assessment, tunnel assay (Roche Applied Science, Indianapolis, Ind.) was preformed according to the manufacturer's instructions and sections were stained for anti-Caspase-3 (Cell Signaling, Beverly, Mass.). For quantification, 3 gels were scored for positive cells.

Immunofluorescence and confocal microscopy. hESC colonies grown on MEFs and HA-hESCs constructs were fixed in situ with accustain (Sigma, St Louis, Mo.) for 20-25 min at room temperature. After blocking with 5% FBS, cells were stained with one of the following primary antibodies: anti-human SSEA4, anti-TRA-1-60, anti-TRA-1-81, anti-Oct3/4, anti-CD44 clone P3H9, anti-Tubulin III isoform (all from Chemicon Temecula, Calif.), anti-CD44 clone A3D8 (Sigma, St Louis, Mo.), anti-CD168 (Novo Castra, Newcastle upon Tyne, UK), anti-CD31, anti-α-fetoprotein (Dako California Inc. Carpinteria, Calif. ), anti-Hyal 1 and Hyal 2 (kindly provided by Inna Gitelman from Ben-Gurion University of the Negev, Israel). Cells were then rinsed three times with PBS (Invitrogen corporation, Carlsbad, Calif.) and incubated for 30 min with suitable FITC-conjugated (R&D systems, Minneapolis, Minn.) or Cy3-conjugated (Sigma, St Louis, Mo.) secondary antibodies. DAPI (2 μg/ml; Sigma, St Louis, Mo.) or To-pro 3 (1:500; Molecular Probe, Invitrogen corporation, Carlsbad, Calif.) were added during the last rinse. IgG isotype-matching using mouse or goat (both from R&D systems, Minneapolis, Minn.) or secondary antibody alone served as controls. The immuno-labeled cells were examined using either fluorescence microscopy (Nikon TE300 inverted microscope) or confocal laser scanning microscopy (Zeiss LSM510 Laser scanning confocal).

HA binding and uptake. The binding assay of fluorescein-labeled hyaluronan was performed as previously described¹⁴. Briefly, hESCs were cultured on coverslips. After gentle washing, human fluorescein-labeled hyaluronan (100 μg/ml, Sigma, St Louis, Mo.) was added to the growth medium for 16 h at 4° C. Following three washes with ice-cold PBS, the cells were fixed in 100% ice-cold acetone for 10 min, air-dried, and then rehydrated 15 min in PBS. Processed cells were further stained with anti-CD44 or anti-CD168 and examined.

RT-PCR. Total RNA was extracted using TriZol® (Gibco Invitrogen Co., San Diego, Calif.), according to manufacturer's instructions. Total RNA was quantified by a UV spectrophotometer and 1 μg was used for each RT sample. One step RT-PCR kit (Qiagen Inc, Valencia, Calif.) was used according to manufacturer's instructions. RT reaction mix was used for negative controls. PCR conditions consisted of: 5 min at 94° C. (hot start), 30-40 cycles (actual number noted below) of: 94° C. for 30 sec, annealing temperature (Ta, noted in Table 1) for 30 sec, 72° C. for 30 sec. A final 7 min extension at 72° C. was performed. Primers used include: HYAL 1 sense 5′GGGCACCTACCCCTACTACACG3′, antisense 5′CATCTGTGACTTCCCTGTGCC3′; HYAL2 sense 5′TGGCCCACGCCTCAAGGTGCC3′, antisense 5′GGCCATGGAGGGCGGAAGCA3′; HYAL3 sense 5′AGCACACTGTGAGGCCCGCTTT3′, antisense 5′GGGGATGTCGGTGCCCAACAA3′; PH20 5′CTTAGTCTCACAGAGGCCAC3′, 5′TACACACTCCTTGCTCCTGG3′. The amplified products were separated on 2% agarose gels containing ethidium bromide.

Karyotyping analysis. Cells were prepared and analyzed as previously described and recommended³⁰. Karyotyping analysis was performed by Dana Faber /Harvard Cancer Research Center, Cytogenetics Laboratory, Cambridge, Mass.

### REFERENCES

1. Xu, C. et al. Feeder-free growth of undifferentiated human embryonic stem cells. Nat Biotechnol. 19, 971-974 (2001).
2. Xu, R. H. et al. Basic FGF and suppression of BMP signaling sustain undifferentiated proliferation of human ES cells. Nat Methods. 2, 185-190 (2005).
3. Richards, M., Fong, C. Y., Chan, W. K., Wong, P. C., Bongso, A. Nat Biotechnol. 20, 933-936 (2002).
4. Amit, M., Shariki, C., Margulets, V., Itskovitz-Eldor, J. Feeder layer- and serum-free culture of human embryonic stem cells Biol Reprod. 70, 837-845 (2004).
5. Stojkovic, P. et al. Human-serum matrix supports undifferentiated growth of human embryonic stem cells. Stem Cells. 2005 May 31; [Epub ahead of print]
6. Proetzel, G., Wiles, M. V. The use of a chemically defined media for the analyses of early development in ES cells and mouse embryos. Methods Mol Biol. 185, 17-26 (2002).
7. Czyz, J., Wobus, A. Embryonic stem cell differentiation: the role of extracellular factors. Differentiation. 68,167-174 (2001).
8. Abbott, A. Cell culture: biology's new dimension. Nature. 424, 870-872 (2003).
9. Yang, S., Leong, K. F., Du, Z., Chua, C. K. The design of scaffolds for use in tissue engineering. Part I. Traditional factors. Tissue Eng 7,679-689 (2001)
10. Toole, B. P. Hyaluronan: from extracellular glue to pericellular cue. Nat Rev Cancer. 4, 528-539 (2004).
11. Toole, B. P. Hyaluronan in morphogenesis. Semin Cell Dev Biol. 12, 79-87 (2001).
12. Tammi, R. et al. Hyaluronan enters keratinocytes by a novel endocytic route for catabolism. J Biol Chem. 276, 35111-35122 (2001).
13. Londbno, I., Bendayan, M. High-resolution cytochemistry of neuraminic and hexuronic acid-containing macromolecules applying the enzyme-gold approach. J Histochem Cytochem. 36, 1005-1014 (1988).
14. S. P., Wight, T. N. Intracellular localization of hyaluronan in proliferating cells. J Histochem Cytochem. 47, 1331-1342 (1999).
15. Campbell, S., et al. CD44 is expressed throughout pre-implantation human embryo development. Hum Reprod. 10, 425-430 (1995).
16. Yang, B., Hall, C. L., Yang, B. L., Savani, R. C., Turley, E. A. Identification of a novel heparin binding domain in RHAMM and evidence that it modifies HA mediated locomotion of ras-transformed cells. J Cell Biochem. 56, 455-468. (1994)
17. Stojkovic, M., et al. Developmental regulation of hyaluronan-binding protein (RHAMM/IHABP) expression in early bovine embryos. Biol Reprod. 68, 60-66 (2003).
18. Stojkovic. M. et al. Effects of high concentrations of hyaluronan in culture medium on development and survival rates of fresh and frozen-thawed bovine embryos produced in vitro. Reproduction. 124, 141-153 (2002).
19. Suh, K. Y., Seong, J., Khademhosseini, A., Laibinis, P. E., Langer, R. A simple soft lithographic route to fabrication of poly(ethylene glycol) microstructures for protein and cell patterning. Biomaterials 25, 557-563 (2004).
20. Burdick, J. A., Chung, C., Jia, X., Randolph, M. A., Langer, R. Controlled degradation and mechanical behavior of photopolymerized hyaluronic acid networks. Biomacromolecules. 6, 386-391 (2005).
21. Hoffman, L. M. Carpenter, M. K. Characterization and culture of human embryonic stem cells. Nat Biotechnol. 23, 699-708 (2005)
22. Carpenter, M. K., Chunhui, Xu., Daight, C. A., Antosiewiczm J. E., Thomsonm J. A. Protocols for the maintenance of human embryonic stem cells. In: Human embryonic stem cells, Chiu, A. Rao, MS. (ed.), Humana Press Inc., Totowa, N.J., 2003.
23. Schatten, G., Smith, J., Navara, C., Park, J. H., Pedersen, R. Culture of human embryonic stem cells. Nat Methods. 2, 455-463 (2005).
24. Heng, B. C., Cao, T., Liu, H., Rufaihah, A. J. Reduced mitotic activity at the periphery of human embryonic stem cell colonies cultured in vitro with mitotically-inactivated murine embryonic fibroblast feeder cells. Cell Biochem Funct. 23, 141-146 (2005).
25. Dang, S., Gerecht-Nir, S., Chen, J., Itskovitz-Eldor, J. Zandstra, P. W. Controlled Scalable Embryonic Stem Cell Differentiation Culture. Stem Cells. 22, 275-282 (2004)
26. Levenberg, S. Huang N F, Lavik E, Rogers A B, Itskovitz-Eldor J, Langer R. Differentiation of human embryonic stem cells on three-dimensional polymer scaffolds. Proc Natl Acad Sci USA. 100, 12741-12746 (2003).
27. McGuire, P. G., Castellot, J. J., Orkin, R. W. Size-dependent hyaluronate degradation by cultured cells. J Cell Physiol. 133, 267-276 (1987)
28. Stern, R. Devising a pathway for hyaluronan catabolism: are we there yet? Glycobiology. 13, 105R-15R (2003).
29. Ferreira, L., Gil, M. H., Dordick, J. S. Enzymatic synthesis of dextran-containing hydrogels. Biomaterials 23, 3957-3967 (2002).
30. Cowan, C. A. Derivation of embryonic stem-cell lines from human blastocysts. N Engl J Med 350, 1353-1356. (2004).

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A composition comprising:
a biocompatible matrix comprising cross-linked hyaluronic acid; and
mammalian embryonic stem cells disposed within the biocompatible matrix, wherein the composition is substantially free of laminin.

2. The composition of claim 1, further comprising a biocompatible aqueous solvent, wherein the concentration of the hyaluronic acid is from about 0.5% to about 1% by weight.

3. The composition of claim 1, further comprising a biocompatible aqueous solvent, wherein the concentration of the hyaluronic acid is from about 1% to about 2% by weight.

4. The composition of claim 1, further comprising a biocompatible aqueous solvent, wherein the concentration of the hyaluronic acid is from about 2% to about 4% by weight.

5. The composition of claim 1, further comprising a biocompatible aqueous solvent, wherein the concentration of the hyaluronic acid is from about 4% to about 6% by weight.

6. The composition of claim 1, further comprising a biocompatible aqueous solvent, wherein the concentration of the hyaluronic acid is from about 6% to about 8% by weight.

7. The composition of claim 1, further comprising a biocompatible aqueous solvent, wherein the concentration of the hyaluronic acid is from about 8% to about 10% by weight.

8. The composition of claim 1, wherein the mammalian embryonic stem cells are human embryonic stem cells.

9. The composition of claim 1, wherein the hyaluronic acid is cross-linked through methacrylate moieties.

10. The composition of claim 1, wherein the hyaluronic acid is crosslinked through acrylate, thiol, or amine groups or through biotin-streptavidin interactions.

11. The composition of claim 1, wherein the density of cells in the composition is from about 5 million cells/mL to about 10 million cells/mL.

12. The composition of claim 1, wherein at least 80% of the embryonic stem cells express one or more of tumor rejecting antigen (TRA), stage specific embryonic antigen-4 (SSEA-4), and Oct 4.

13. The composition of claim 1, wherein at least 85% of the embryonic stem cells express one or more of tumor rejecting antigen (TRA), stage specific embryonic antigen-4 (SSEA-4), and Oct 4.

14. The composition of claim 1, wherein at least 90% of the embryonic stem cells express one or more of tumor rejecting antigen (TRA), stage specific embryonic antigen-4 (SSEA-4), and Oct 4.

15. The composition of claim 1, wherein at least 95% of the embryonic stem cells express one or more of tumor rejecting antigen (TRA), stage specific embryonic antigen-4 (SSEA-4), and Oct 4.

16. The composition of claim 1, wherein at most 10% of the embryonic stem cells express one or more of CD31, alpha-fetoprotein, and tubulin.

17. The composition of claim 1, wherein at most 5% of the embryonic stem cells express one or more of CD31, alpha-fetoprotein, and tubulin.

18. The composition of claim 1, wherein at most 1% of the embryonic stem cells express one or more of CD31, alpha-fetoprotein, and tubulin.

19. The composition of claim 1, wherein the cells encapsulated within the biocompatible matrix maintain a stable phenotype in culture at least 30 doublings.

20. The composition of claim 1, wherein the cells encapsulated within the biocompatible matrix maintain a stable phenotype in culture for at least 30 days.

21. The composition of claim 1, wherein the cells encapsulated within the biocompatible matrix maintain a stable phenotype in culture for at least 40 days.

22. The composition of claim 1, wherein the molecular weight of the hyaluronic acid is about 50 kDa, about 350 kDa, or about 1100 kDa.

23. The composition of claim 1, wherein the molecular weight of the hyaluronic acid is from about 5 kDa to about 50 kDa.

24. The composition of claim 1, wherein the molecular weight of the hyaluronic acid is from about 50 kDa to about 100 kDa.

25. The composition of claim 1, wherein the molecular weight of the hyaluronic acid is from about 100 kDa to about 500 kDa.

26. The composition of claim 1, wherein the molecular weight of the hyaluronic acid is from about 500 kDa to about 1000 kDa.

27. The composition of claim 1, wherein the molecular weight of the hyaluronic acid is from about 1000 kDa to about 1500 kDa.

28. The composition of claim 1, wherein the molecular weight of the hyaluronic acid is from about 1500 kDa to about 2000 kDa.

29. The composition of claim 1, wherein the biocompatible aqueous solvent is culture media.

30. A composition, comprising:
a biocompatible matrix consisting essentially of cross-linked hyaluronic acid;
mammalian embryonic stem cells disposed within the biocompatible matrix; and
a biocompatible aqueous solvent.

31. The composition of claim 30, further comprising a biocompatible aqueous solvent, wherein the concentration of the hyaluronic acid is from about 0.5% to about 1% by weight.

32. The composition of claim 30, further comprising a biocompatible aqueous solvent, wherein the concentration of the hyaluronic acid is from about 1% to about 2% by weight.

33. The composition of claim 30, further comprising a biocompatible aqueous solvent, wherein the concentration of the hyaluronic acid is from about 2% to about 4% by weight.

34. The composition of claim 30, further comprising a biocompatible aqueous solvent, wherein the concentration of the hyaluronic acid is from about 4% to about 6% by weight.

35. The composition of claim 30, further comprising a biocompatible aqueous solvent, wherein the concentration of the hyaluronic acid is from about 6% to about 8% by weight.

36. The composition of claim 30, further comprising a biocompatible aqueous solvent, wherein the concentration of the hyaluronic acid is from about 8% to about 10% by weight.

37. The composition of claim 30, wherein the mammalian embryonic stem cells are human embryonic stem cells.

38. The composition of claim 30, wherein the hyaluronic acid is crosslinked through methacrylate moieties.

39. The composition of claim 30, wherein the hyaluronic acid is crosslinked through acrylate, thiol, or amine groups or through biotin-streptavidin interactions.

40. The composition of claim 30, wherein the density of cells in the composition is from about 5 million cells/mL to about 10 million cells/mL.

41. The composition of claim 30, wherein at least 80% of the embryonic stem cells express one or more of tumor rejecting antigen (TRA), stage specific embryonic antigen-4 (SSEA-4), and Oct 4.

42. The composition of claim 30, wherein at most 10% of the embryonic stem cells express one or more of CD31, alpha-fetoprotein, and tubulin.

43. The composition of claim 30, wherein the cells encapsulated within the biocompatible matrix maintain a stable phenotype in culture at least 30 doublings.

44. The composition of claim 30, wherein the cells encapsulated within the biocompatible matrix maintain a stable phenotype in culture for at least 30 days.

45. The composition of claim 30, wherein the cells encapsulated within the biocompatible matrix maintain a stable phenotype in culture for at least 40 days.

46. The composition of claim 30, wherein the molecular weight of the hyaluronic acid is about 50 kDa, about 350 kDa, or about 1100 kDa.

47. The composition of claim 30, wherein the molecular weight of the hyaluronic acid is from about 5 kDa to about 2000 kDa.

48. The composition of claim 30, wherein the biocompatible aqueous solvent is culture media.

49. A composition comprising:
a biocompatible matrix comprising cross-linked hyaluronic acid;
mammalian embryonic stem cells disposed within the biocompatible matrix; and
a biocompatible aqueous solvent,
wherein the concentration of the hyaluronic acid in the solvent is greater than 1.5% by weight.

50. The composition of claim 49, wherein the concentration is greater than about 2.0% by weight.

51. The composition of claim 49, wherein the concentration of the hyaluronic acid is greater than about 3% by weight.

52. The composition of claim 49, wherein the concentration of the hyaluronic acid is greater than about 4% by weight.

53. The composition of claim 49, wherein the concentration of the hyaluronic acid is greater than about 5% by weight.

54. The composition of claim 49, wherein the mammalian embryonic stem cells are human embryonic stem cells.

55. The composition of claim 49, wherein the hyaluronic acid is crosslinked through methacrylate moieties.

56. The composition of claim 49, wherein the hyaluronic acid is crosslinked through acrylate, thiol, or amine groups or through biotin-streptavidin interactions.

57. The composition of claim 49, wherein the density of cells in the composition is from about 5 million cells/mL to about 10 million cells/mL.

58. The composition of claim 49, wherein at least 80% of the embryonic stem cells express one or more of tumor rejecting antigen (TRA), stage specific embryonic antigen-4 (SSEA-4), and Oct 4.

59. The composition of claim 49, wherein at least 85% of the embryonic stem cells express one or more of tumor rejecting antigen (TRA), stage specific embryonic antigen-4 (SSEA-4), and Oct 4.

60. The composition of claim 49, wherein at least 90% of the embryonic stem cells express one or more of tumor rejecting antigen (TRA), stage specific embryonic antigen-4 (SSEA-4), and Oct 4.

61. The composition of claim 49, wherein at least 95% of the embryonic stem cells express one or more of tumor rejecting antigen (TRA), stage specific embryonic antigen-4 (SSEA-4), and Oct 4.

62. The composition of claim 49, wherein at most 10% of the embryonic stem cells express one or more of CD31, alpha-fetoprotein, and tubulin.

63. The composition of claim 49, wherein at most 5% of the embryonic stem cells express one or more of CD31, alpha-fetoprotein, and tubulin.

64. The composition of claim 49, wherein at most 1% of the embryonic stem cells express one or more of CD31, alpha-fetoprotein, and tubulin.

65. The composition of claim 49, wherein the cells encapsulated within the biocompatible matrix maintain a stable phenotype in culture at least 30 doublings.

66. The composition of claim 49, wherein the cells encapsulated within the biocompatible matrix maintain a stable phenotype in culture for at least 30 days.

67. The composition of claim 49, wherein the cells encapsulated within the biocompatible matrix maintain a stable phenotype in culture for at least 40 days.

68. A method of culturing embryonic stem cells, comprising:
providing a population of embryonic stem cells;
combining the embryonic stem cells with hyaluronic acid to form a mixture; and
causing the hyaluronic acid to cross-link in a solvent, thereby encapsulating the embryonic stem cells in a hyaluronic acid hydrogel.

69. The method of claim 68, further comprising culturing the encapsulated embryonic stem cells in vitro.

70. The method of claim 68, further comprising maintaining the embryonic stem cells in culture for at least 30 days, and wherein the cells maintain a stable phenotype.

71. The method of claim 69, further comprising maintaining the embryonic stem cells in culture for at least 40 days, and wherein the cells maintain a stable phenotype.

72. The method of claim 69, further comprising maintaining the embryonic stem cells in culture for at least 30 doublings, and wherein the cells maintain a stable phenotype.

73. The method of claim 68, wherein causing comprises promoting radical chain polymerization, ionic chain polymerization, or step polymerization.

74. The method of claim 68, wherein the hyaluronic acid is terminated with methacrylate groups.

75. The method of claim 68, wherein the hyaluronic acid is terminated with acrylate groups, thiols, or amines.

76. The method of claim 68, wherein the molecular weight of the hyaluronic acid is about 50 kDa, about 350 kDa, or about 1100 kDa.

77. The method of claim 68, wherein the molecular weight of the hyaluronic acid is from about 5 kDa to about 50 kDa.

78. The method of claim 68, wherein the molecular weight of the hyaluronic acid is from about 50 kDa to about 100 kDa.

79. The method of claim 68, wherein the molecular weight of the hyaluronic acid is from about 100 kDa to about 500 kDa.

80. The method of claim 68, wherein the molecular weight of the hyaluronic acid is from about 500 kDa to about 1000 kDa.

81. The method of claim 68, wherein the molecular weight of the hyaluronic acid is from about 1000 kDa to about 1500 kDa.

82. The method of claim 68, wherein the molecular weight of the hyaluronic acid is from about 1500 kDa to about 2000 kDa.

83. The method of claim 68, further comprising allowing the cells to proliferate, releasing the cells from the hydrogel, dividing the cells into a plurality of populations, and repeating the method of claim 68 with each population in the plurality of populations.

84. The method of claim 68, further comprising contacting the hydrogel with hyaluronidase to release the embryonic stem cells.

85. A method of producing a population of embryonic stem cells, comprising:
providing a population of mammalian embryonic stem cells;
combining the embryonic stem cells with methacrylate-terminated hyaluronic acid;
causing the hyaluronic acid to cross-link in a solvent, thereby encapsulating the embryonic stem cells in a hyaluronic acid hydrogel; and
contacting the hydrogel with hyaluronidase to release the embryonic stem cells.

86. The method of claim 85, further comprising, before contacting the hydrogel, culturing the encapsulated embryonic stem cells in vitro.

87. The method of claim 85, further comprising repeating combining and causing with the released embryonic stem cells.
